(19) United States
(12) Patent Application Publication
(10) Pub. No.: US 2025/0213849 A1
(43) Pub. Date: Jul. 3, 2025

(54) Electrode Assembly for Delivering Alternating Electric Fields (e.g., TTFields) with Peripherally-Positioned Temperature Sensors

(71) Applicant: Novocure GmbH, Root D4 (CH)

(72) Inventors: Yoram WASSERMAN, Haifa (IL); Stas OBUCHOVSKY, Haifa (IL); Nataliya KUPLENNIK, Haifa (IL); David SHAPIRO, Haifa (IL); Elie YAACOBI, Haifa (IL); Noa HALAVEE, Haifa (IL); Dmitry GOLOM, Haifa (IL)

(73) Assignee: Novocure GmbH, Root D4 (CH)

(21) Appl. No.: 19/003,063

(22) Filed: Dec. 27, 2024

Related U.S. Application Data
(60) Provisional application No. 63/615,909, filed on Dec. 29, 2023.
(60) Provisional application No. 63/615,891, filed on Dec. 29, 2023.

(51) Int. Cl. A61N 1/04; A61N 1/36

(52) U.S. Cl. CPC A61N 1/0408; A61N 1/0496; A61N 1/36002; A61N 1/36014

(57) ABSTRACT

Alternating electric fields (e.g., Tumor Treating Fields or TTFields) can be applied to a subject's body using an apparatus (e.g., a transducer array) that includes at least one metal pad disposed on a substrate and a plurality of temperature sensors (e.g., thermistors). The temperature sensors are disposed on the substrate at positions that are outside a first convex hull that encloses the at least one metal pad (e.g., at the edges of the apparatus). A layer of graphite is disposed in front of the metal pads and the temperature sensors and is in thermal contact with both the metal pads and the temperature sensors. Positioning the thermistors outside the first convex hull (e.g., at the edges of the apparatus) can help prevent overheating in situations where the edges of the apparatus tend to be the hottest portions of the apparatus.

### CROSS REFERENCE TO RELATED APPLICATIONS

This Application claims the benefit of U.S. Provisional Application 63/615,909, filed Dec. 29, 2023, and also U.S. Provisional Application 63/615,891, filed Dec. 29, 2023, each of which is incorporated herein by reference in its entirety.

### BACKGROUND

Tumor Treating Fields (TTFields) therapy is a proven approach for treating tumors using alternating electric fields at frequencies e.g., between 50 kHz-5 MHz, more commonly 100-500 KHz. The alternating electric fields are induced by electrode assemblies (e.g., arrays of capacitively coupled electrodes, also called transducer arrays) placed on the subject's skin on opposite sides of the subject's body. When an AC voltage is applied between opposing electrode assemblies, an AC current is coupled through the electrode assemblies and into the subject's body. And higher currents are strongly correlated with higher efficacy of treatment.

Alternating electric fields can also be used to treat medical conditions other than tumors. For example, as described in U.S. Pat. No. 10,967,167, alternating electric fields e.g., at 75-150 kHz can be used to increase the permeability of the blood brain barrier (BBB) so that, e.g., chemotherapy drugs can reach the brain.

One example of prior art electrode assemblies that can be used to apply alternating electric fields to a subject's body are the electrode assemblies described in U.S. Pat. No. 8,715,203. These electrode assemblies include nine electrode elements, each of which includes (a) a metal layer and (b) a ceramic layer with a very high dielectric constant positioned between the metal layer and the subject's skin. Thermistors positioned in small holes in the center of most of the electrode elements are included to make temperature measurements at those electrode elements.

Another example of prior art electrode assemblies that can be used to apply alternating electric fields to a subject's body are the electrode assemblies described in Pub. No. US 2021/0402179. These electrode assemblies have a flex circuit that includes a plurality of conductive pads on the front side of the flex circuit and a plurality of flexible polymer regions disposed over and in front of the conductive pads. A plurality of thermistors positioned on a rear side of the flex circuit in thermal contact with respective conductive pads are used to sense the temperature of the conductive pads. In alternative embodiments described in US 2021/0402179, the thermistors are positioned between the conductive pads.

To use either of these prior art electrode assemblies, an AC voltage is applied to the metal layers of the electrode elements in opposing electrode assemblies to generate the TTFields in the subject's body. The skin beneath the electrode elements heats up during use, resulting in a set of hotter spots immediately below the electrode elements, and a set of cooler regions immediately below the spaces between the electrode elements. And because safety considerations require that the skin temperature remains below a safety threshold (e.g., 41° C.), these hot spots limit the amount of current that can be delivered through the prior art electrode assemblies. More specifically, the prior art systems rely on signals from the thermistors to ensure that the current is low enough to prevent the temperature of the subject's skin from exceeding the safety threshold.

### SUMMARY OF THE INVENTION

One aspect of this application is directed to a first apparatus for applying an electrical signal to a subject's body. The first apparatus comprises a substrate, at least one metal pad disposed on the substrate, a plurality of temperature sensors, and a layer of graphite. Each of the temperature sensors is disposed on the substrate at a position that is outside a first convex hull that encloses the at least one metal pad. The layer of graphite is disposed in front of the at least one metal pad and in front of the plurality of temperature sensors, and the layer of graphite has an area that is at least as large as a second convex hull that encloses the at least one metal pad and the plurality of temperature sensors. The layer of graphite is positioned to be in thermal contact with (a) each of the plurality of temperature sensors and (b) the at least one metal pad.

In some embodiments of the first apparatus, the layer of graphite comprises a layer of pyrolytic graphite, graphitized polymer film, or graphite foil made from compressed high purity exfoliated mineral graphite.

Some embodiments of the first apparatus further comprise a first layer of conductive adhesive or conductive gel disposed on and behind the layer of graphite, and a second layer of conductive adhesive or conductive gel disposed on and in front of the layer of graphite.

Some embodiments of the first apparatus further comprise a layer of dielectric material disposed on the at least one metal pad, between the at least one metal pad and the layer of graphite. In these embodiments, the thermal contact between the at least one metal pad and the layer of graphite traverses the layer of dielectric material, and the layer of dielectric material has a dielectric constant of at least 10. Optionally, in these embodiments, the layer of dielectric material comprises a polymer film. Optionally, in these embodiments, the layer of dielectric material comprises a ceramic material.

In some embodiments of the first apparatus, the substrate is a single PCB substrate, the at least one metal pad comprises at least one PCB pad, and the plurality of temperature sensors are mounted to the single PCB substrate.

In some embodiments of the first apparatus, the substrate comprises a first PCB having a first PCB substrate and a second PCB having a second PCB substrate, the at least one metal pad comprises at least one PCB pad of the first PCB, and the plurality of temperature sensors are mounted to the second PCB substrate.

In some embodiments of the first apparatus, each of the metal pads is a copper pad or a stainless steel pad.

Some embodiments of the first apparatus further comprise a flexible backing positioned behind the substrate, and the flexible backing is configured to support the substrate, and at least a portion of the flexible backing extends laterally beyond the substrate. Optionally, in these embodiments, at least a portion of the flexible backing is covered with a biocompatible adhesive that adheres to skin.

In some embodiments of the first apparatus, each of the plurality of temperature sensors comprises a thermistor. In some embodiments of the first apparatus, each of the plurality of temperature sensors is positioned near an edge of the layer of graphite.

Another aspect of this application is directed to a second apparatus for applying an electrical signal to a subject's body. The second apparatus comprises a flexible PCB, a plurality of temperature sensors, a first layer of conductive adhesive or conductive gel, and a flexible backing. The flexible PCB has at least one metal pad disposed on the PCB and a plurality of conductive traces disposed on the PCB. Each of the plurality of temperature sensors is mounted to the PCB and positioned outside a first convex hull that encloses the at least one metal pad, and each of the plurality of temperature sensors is (a) disposed in indirect thermal contact with the at least one metal pad and (b) electrically connected to at least one of the conductive traces of the PCB. The first layer of conductive adhesive or conductive gel is disposed on and in front of the PCB. And the flexible backing is positioned behind the PCB. The flexible backing is configured to support the PCB, and at least a portion of the flexible backing extends laterally beyond the PCB.

Some embodiments of the second apparatus further comprise a layer of anisotropic material disposed on and in front of the first layer of conductive adhesive or conductive gel. In these embodiments, the layer of anisotropic material has an area that is at least as large as a second convex hull that encloses the at least one metal pad and the plurality of temperature sensors. In addition, a second layer of conductive adhesive or conductive gel is disposed on and in front of the layer of anisotropic material, and the layer of anisotropic material facilitates the indirect thermal contact between the plurality of temperature sensors and the at least one metal pad.

Optionally, in the embodiments described in the previous paragraph, the layer of anisotropic material comprises graphite. Optionally, in the embodiments described in the previous paragraph, the layer of anisotropic material comprises a layer of pyrolytic graphite, graphitized polymer film, or graphite foil made from compressed high purity exfoliated mineral graphite. Optionally, in the embodiments described in the previous paragraph, each of the plurality of temperature sensors is positioned near an edge of the layer of anisotropic material.

Some embodiments of the second apparatus further comprise a layer of dielectric material disposed on the at least one metal pad and in front of the at least one metal pad. In these embodiments, the indirect thermal contact between the plurality of temperature sensors and the at least one metal pad traverses the layer of dielectric material, and the layer of dielectric material has a dielectric constant of at least 10. Optionally, in these embodiments, the layer of dielectric material comprises a polymer film. Optionally, in these embodiments, the layer of dielectric material comprises a ceramic material.

In some embodiments of the second apparatus, each of the metal pads is a copper pad or a stainless steel pad. In some embodiments of the second apparatus, at least a portion of the flexible backing is covered with a biocompatible adhesive that adheres to skin. In some embodiments of the second apparatus, each of the plurality of temperature sensors comprises a thermistor. In some embodiments of the second apparatus, the flexible PCB has a central region and a plurality of stalk-shaped extensions that extend from the central region, and the plurality of temperature sensors are mounted on the stalk-shaped extensions.

Another aspect of this application is directed to a third apparatus for applying an electrical signal to a subject's body. The third apparatus has a front face facing the subject's body, and it comprises a substrate, at least one metal pad, a plurality of temperature sensors, and a layer of graphite. The at least one metal pad is disposed on the substrate. When viewed from a direction perpendicular to the front face of the apparatus, at least one of the plurality of temperature sensors is disposed on the substrate at a position that is outside a first convex hull that encloses the at least one metal pad. The layer of graphite is disposed in front of the at least one metal pad and in front of the plurality of temperature sensors. The layer of graphite has an area that is at least as large as a second convex hull that encloses the at least one metal pad and the plurality of temperature sensors, and the layer of graphite is positioned to be in thermal contact with (a) each of the plurality of temperature sensors and (b) the at least one metal pad.

In some embodiments of the third apparatus, each of the plurality of temperature sensors is disposed on the substrate at a position that is outside the first convex hull. In some embodiments of the third apparatus, the layer of graphite comprises a layer of pyrolytic graphite, graphitized polymer film, or graphite foil made from compressed high purity exfoliated mineral graphite.

Some embodiments of the third apparatus further comprise a first layer of conductive adhesive or conductive gel disposed on and behind the layer of graphite, and a second layer of conductive adhesive or conductive gel disposed on and in front of the layer of graphite.

Some embodiments of the third apparatus further comprise a layer of dielectric material disposed on the at least one metal pad, between the at least one metal pad and the layer of graphite. In these embodiments, the thermal contact between the at least one metal pad and the layer of graphite traverses the layer of dielectric material, and the layer of dielectric material has a dielectric constant of at least 10. Optionally, in these embodiments, the layer of dielectric material can comprise a polymer film.

In some embodiments of the third apparatus, the substrate is a single PCB substrate, the at least one metal pad comprises at least one PCB pad, and the plurality of temperature sensors are mounted to the single PCB substrate.

In some embodiments of the third apparatus, the substrate comprises a first PCB having a first PCB substrate and a second PCB having a second PCB substrate, the at least one metal pad comprises at least one PCB pad of the first PCB, and the plurality of temperature sensors are mounted to the second PCB substrate.

Some embodiments of the third apparatus further comprise a flexible backing positioned behind the substrate, and the flexible backing is configured to support the substrate.

In some embodiments of the third apparatus, each of the plurality of temperature sensors comprises a thermistor. In some embodiments of the third apparatus, at least one, optionally all, of the plurality of temperature sensors is positioned within 2 cm of an edge of the layer of graphite. In some embodiments of the third apparatus, the substrate comprises a PCB substrate having one or more central regions, and each of the plurality of temperature sensors is located outside the first convex hull and is disposed on a respective protrusion that extends from the one or more central regions of the PCB substrate.

Another aspect of this application is directed to a fourth apparatus for applying an electrical signal to a subject's body. The fourth apparatus has a front face facing the subject's body, and it comprises a flexible PCB, a plurality of temperature sensors, a first layer of conductive adhesive or conductive gel, and a flexible backing. The flexible PCB has at least one metal pad disposed on the PCB and a plurality of conductive traces disposed on the PCB. When viewed from a direction perpendicular to the front face of the apparatus, at least one of the plurality of temperature sensors is mounted to the PCB and positioned outside a first convex hull that encloses the at least one metal pad. Each of the plurality of temperature sensors is (a) disposed in indirect thermal contact with the at least one metal pad and (b) electrically connected to at least one of the conductive traces of the PCB. The first layer of conductive adhesive or conductive gel is disposed on and in front of the PCB. The flexible backing is positioned behind the PCB, and is configured to support the PCB.

In some embodiments of the fourth apparatus, each of the plurality of temperature sensors is mounted to the PCB and positioned outside the first convex hull.

Some embodiments of the fourth apparatus further comprise a layer of anisotropic material and a second layer of conductive adhesive or conductive gel. In these embodiments, the layer of anisotropic material is disposed on and in front of the first layer of conductive adhesive or conductive gel, and the layer of anisotropic material has an area that is at least as large as a second convex hull that encloses the at least one metal pad and the plurality of temperature sensors. The second layer of conductive adhesive or conductive gel is disposed on and in front of the layer of anisotropic material. And the layer of anisotropic material facilitates the indirect thermal contact between the plurality of temperature sensors and the at least one metal pad.

Optionally, in the embodiments described in the previous paragraph, the layer of anisotropic material comprises graphite. Optionally, in the embodiments described in the previous paragraph, at least one, optionally all, of the plurality of temperature sensors is positioned within 2 cm of an edge of the layer of anisotropic material. Optionally, in the embodiments described in the previous paragraph, the flexible PCB has a central region and a plurality of protrusions that extend from the central region, and the plurality of temperature sensors are mounted on the protrusions.

In some embodiments of the fourth apparatus, the flexible PCB has a central region and a plurality of protrusions that extend from the central region, and the plurality of temperature sensors are mounted on the protrusions.

Some embodiments of the fourth apparatus further comprise a layer of dielectric material disposed on the at least one metal pad and in front of the at least one metal pad. In these embodiments, the indirect thermal contact between the plurality of temperature sensors and the at least one metal pad traverses the layer of dielectric material, and the layer of dielectric material has a dielectric constant of at least 10. Optionally, in these embodiments, the layer of dielectric material can comprise a polymer film.

In some embodiments of the fourth apparatus, at least a portion of the flexible backing is covered with a biocompatible adhesive that adheres to skin. In some embodiments of the fourth apparatus, each of the plurality of temperature sensors comprises a thermistor. In some embodiments of the fourth apparatus, the flexible PCB has one or more central regions and one or more protrusions that extend from one or more of the one or more central regions, and a temperature sensor is mounted on the one or more protrusions.

Another aspect of this application is directed to a fifth apparatus for applying an electrical signal to a subject's body. The fifth apparatus has a front face facing the subject's body, and it comprises a flexible PCB, a plurality of temperature sensors, a first layer of conductive adhesive or conductive gel, a layer of graphite, a second layer of conductive adhesive or conductive gel, and a flexible backing. The flexible PCB includes at least one metal pad positioned on at least one main portion of the PCB, and at least one conductive trace that protrudes from the at least one main portion of the PCB. At least one of the plurality of temperature sensors is (a) disposed in indirect thermal contact with the at least one metal pad, (b) positioned at a distal end of at least one of the conductive traces that protrudes from the at least one main portion of the PCB, and (c) electrically connected to at least one of the conductive traces that protrudes from the at least one main portion of the PCB. The first layer of conductive adhesive or conductive gel is disposed on and in front of the PCB. The layer of graphite is disposed on and in front of the first layer of conductive adhesive or conductive gel, and the layer of graphite facilitates the indirect thermal contact between the plurality of temperature sensors and the at least one metal pad. The second layer of conductive adhesive or conductive gel is disposed on and in front of the layer of graphite. The flexible backing is positioned behind the PCB and is configured to support the PCB.

In some embodiments of the fifth apparatus, at least one of the conductive traces to which a temperature sensor is affixed protrudes in an outward direction from the at least one main portion of the PCB. In some embodiments of the fifth apparatus, at least one of the conductive traces to which a temperature sensor is affixed protrudes in an inward direction from the at least one main portion of the PCB.

### BRIEF DESCRIPTION OF THE DRAWINGS

FIG. 1 is a plan view of an apparatus for applying alternating electric fields (e.g., TTFields) to a subject's body.

FIG. 2 is a section view of the apparatus of FIG. 1.

FIG. 3 depicts an example of how to use the apparatus of FIG. 1 to apply alternating electric fields (e.g., TTFields) to a subject's body.

FIG. 4 is a plan view of another apparatus for applying alternating electric fields (e.g., TTFields) to a subject's body.

FIG. 5 is a section view of the apparatus of FIG. 4.

FIG. 6 depicts another apparatus for applying alternating electric fields (e.g., TTFields) to a subject's body.

FIG. 7 is a section view of the apparatus of FIG. 6.

FIG. 8 depicts another apparatus for applying alternating electric fields (e.g., TTFields) to a subject's body.

FIG. 9 is a section view of the apparatus of FIG. 8.

FIG. 10 depicts another apparatus for applying alternating electric fields (e.g., TTFields) to a subject's body.

FIG. 11 depicts another apparatus for applying alternating electric fields (e.g., TTFields) to a subject's body.

Various embodiments are described in detail below with reference to the accompanying drawings, wherein like reference numerals represent like elements.

### DESCRIPTION OF THE PREFERRED EMBODIMENTS

Notably, in the two prior art designs described above, the hottest spots are always located directly beneath one of the electrode elements. It is therefore reasonable for these prior art systems to rely on temperature readings obtained at the electrode elements. But with other designs for transducer arrays, obtaining temperature measurements only at the locations of the electrode elements may be insufficient to ensure that no portions of the transducer array heat up beyond the safety threshold. This can be particularly important in designs that use sheets of heat-conductive materials (e.g., graphite) to spread the heat out within any given transducer array. Examples of this type of transducer array are described in Pub. No. US 2023/0043071, which is incorporated herein by reference in its entirety. And in these transducer arrays, the peripheral portions (i.e., portions near an edge of a transducer array) can sometimes run hotter than the portions that sit directly beneath the electrode elements.

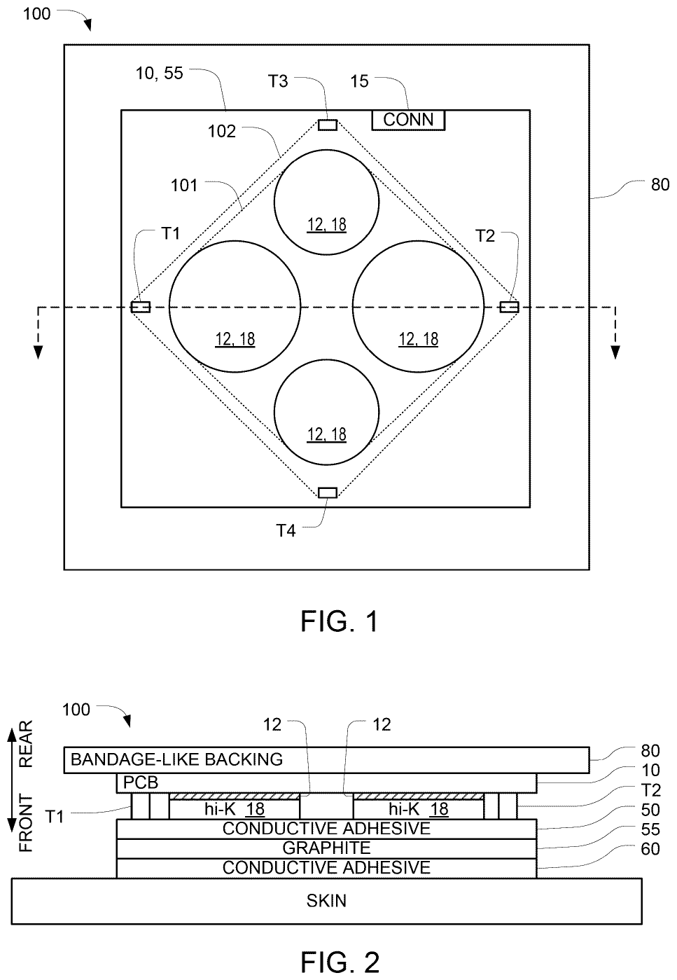

FIG. 1 is a plan view of an apparatus 100 (e.g., a transducer array) for applying alternating electric fields (e.g., TTFields) to a subject's body, and FIG. 2 is a section view of the same apparatus 100. The apparatus 100 includes a substrate (e.g., a PCB) 10 and at least one metal pad 12 disposed on the substrate. Suitable materials for the at least one metal pad 12 include, e.g., copper and stainless steel. Note that as used herein, the term “PCB” refers to a printed circuit board, and this term encompasses rigid PCBs (e.g., with copper traces on a rigid epoxy board), flex circuits (e.g., with copper traces on a flexible polyimide substrate), and printed circuits made by printing a conductive ink on a flexible substrate.

In geometry, a convex hull of a set of objects is the smallest convex polygon that encloses all of the objects. A first convex hull 101 (depicted using dotted lines) encloses the at least one metal pad 12. A plurality of temperature sensors (e.g., thermistors) T1-T4 are disposed on the substrate PCB 10 at positions that are *outside* the first convex hull 101. These thermistors are used to obtain temperature readings of portions of the apparatus 100 that are located peripherally with respect to the metal pads 12. A second convex hull 102 encloses the at least one metal pad 12 and the plurality of temperature sensors T1-T4. Note also that not all of the temperature sensors must be positioned outside the first convex hull, as depicted in FIG. 1. To the contrary—some of the temperature sensors can be positioned inside the first convex hull, as long as at least one of the temperature sensors is positioned outside the first convex hull.

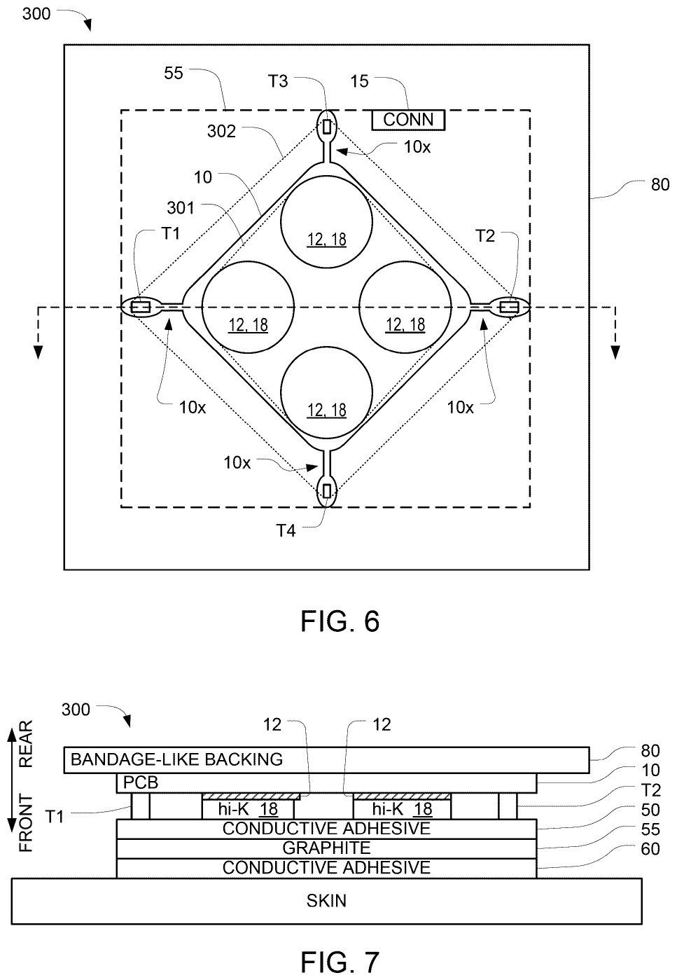

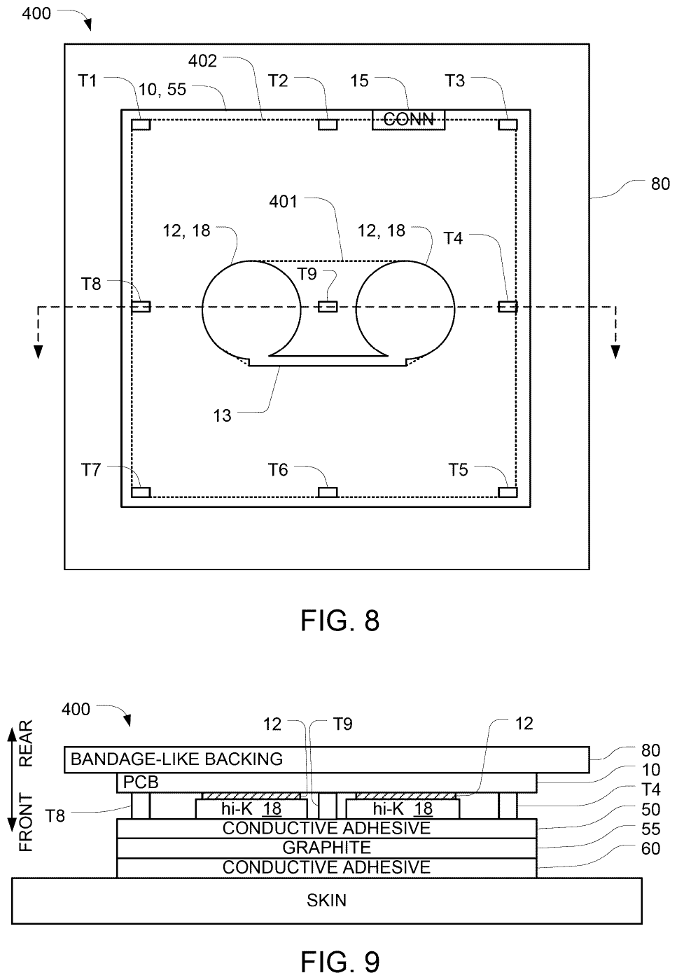

A layer of graphite 55 is disposed in front of the at least one metal pad 12 and in front of the plurality of temperature sensors T1-T4, and this layer of graphite 55 has an area that is at least as large as the second convex hull 102. Although drawn to be coincident in FIGS. 1-2, in some embodiments, the layer of graphite 55 may have an area that is larger than that of the PCB 10 (as shown, for example, in FIGS. 6-7 and 10. The layer of graphite 55 is positioned to be in thermal contact with (a) each of the plurality of temperature sensors T1-T4 and (b) the at least one metal pad 12. The layer of graphite 55 (e.g., a sheet of graphite) could be made from, e.g., pyrolytic graphite, graphitized polymer film, or graphite foil made from compressed high purity exfoliated mineral graphite. The layer of graphite 55 is both thermally conductive and electrically conductive, and it acts to spread both the flow of current and heat in all four directions (i.e., to the right, to the left, into the page, and out of the page in FIG. 2, which corresponds to right, left, up, and down in FIG. 1). In some embodiments, each of the plurality of temperature sensors T1-T4 is positioned near an edge of the layer of graphite 55. For example, each of the plurality of temperature sensors T1-T4 can be positioned within 2 cm of the edge of the layer of graphite 55, or within 1 cm, or within 5 mm, or within 3 mm of the edge of the layer of graphite 55. (Note that this does not preclude additional temperature sensors other than T1-T4 from being positioned at different locations; see, for example FIG. 8.)

In the embodiment depicted in FIGS. 1-2, a connector 15 is mounted to the substrate PCB 10, and this connector 15 is used to provide an electrical interface with the thermistors T1-T4 and the at least one metal pad 12. When more than one metal pad 12 is included (as depicted in FIGS. 1-2), all the metal pads 12 can be connected by conductive traces (not shown), in which case only a single pin of the connector 15 will be required to apply an AC signal to all of the metal pads 12. In alternative embodiments, each metal pad 12 can be electrically connected to its own individual pin on the connector 15.

In the embodiment depicted in FIGS. 1-2, the substrate PCB 10 also includes a layer of dielectric material 18 disposed on and in front of the metal pads 12, between the at least one metal pad 12 and the layer of graphite 55. This layer of dielectric material 18 has a dielectric constant of at least 10, and it could be, for example, a polymer film or a layer of a ceramic material. When the layer of dielectric material 18 is present (as depicted in FIGS. 1-2), the thermal contact between the at least one metal pad 12 and the layer of graphite 55 traverses the layer of dielectric material 18. In some preferred embodiments, the layer of dielectric material 18 has a dielectric constant of at least 20 or at least 40.

A first layer of conductive adhesive 50 can be disposed on and behind the layer of graphite 55. In FIGS. 1-2, the first layer of conductive adhesive 50 is shown disposed on and behind the layer of graphite 55, and the rear surface of this conductive adhesive 50 adheres to the front surface of the dielectric material 18 and to the front surfaces of the thermistors T1-T4 and may also contact some portion(s) of the PCB 10. Note that in some embodiments, the layer of dielectric material 18 is not included, in which case the rear surface of the first layer of conductive adhesive 50 will adhere to the front surface of the metal pads 12 and to the front surfaces of the thermistors T1-T4 and may also contact some portion(s) of the PCB 10. Note that in alternative embodiments, a layer of conductive gel (e.g., hydrogel) can be used in place of the first layer of conductive adhesive 50 depicted in FIG. 2.

A flexible backing 80 (e.g., a bandage-like backing) is positioned behind the substrate PCB 10, and this flexible backing 80 is configured to support the substrate PCB. In the embodiment depicted in FIGS. 1-2, a portion of the flexible backing 80 extends laterally beyond the substrate PCB 10 and the front of this portion is covered with a biocompatible adhesive that adheres to skin. This portion of the flexible backing 80 helps hold the apparatus 100 against the subject's skin. In other embodiments (not shown), the flexible backing does not extend laterally beyond the substrate PCB 10, and the apparatus 100 may be supported in position on the subject's skin by an adhesive present on the front face of the apparatus (e.g., such as via conductive adhesive 60 or an adhesive coated foam perimeter).

The embodiment depicted in FIGS. 1-2 also includes a second layer of conductive adhesive 60 disposed on and in front of the layer of graphite 55. The second layer of conductive adhesive 60 should be biocompatible, and its function is to hold the apparatus 100 against the subject's skin. Note that in alternative embodiments, a layer of conductive gel (e.g., hydrogel) can be used in place of the second layer of conductive adhesive 60 depicted in FIG. 2.

Note that in the embodiment depicted in FIGS. 1 and 2, a single PCB serves as the substrate 10. In this case, the at least one metal pad 12 would be one or more metal pads on the single PCB, and the plurality of temperature sensors would be mounted to the single PCB. But in alternative embodiments, two or more structures could collectively serve as the substrate. In one example, the substrate can be the combination of (a) a first PCB having a first PCB substrate, (b) a second PCB having a second PCB substrate, and, optionally, (c) a single piece of material upon which both the first and second PCBs are mounted. In this example, the at least one metal pad would be pad(s) of the first PCB, and the plurality of temperature sensors are mounted to the second PCB substrate.

Positioning the temperature sensors T1-T4 outside the first convex hull 101 is particularly advantageous in embodiments that include the layer of graphite material 55, because unlike the prior art embodiments (in which it is virtually certain that the hottest spot will be directly beneath an electrode element), embodiments that include a layer of graphite material 55 spread out the current and heat over a much larger surface. And as a result, additional factors, including but not limited to geometric factors and anatomical factors, may impact which portion of the apparatus 100 will be the hottest. In one example, geometry comes into play because peripheral portions of opposing transducer arrays that are closest to each other will tend to run hotter than the central portions of those transducer arrays (which are spaced further apart). In this case, positioning the temperature sensors T1-T4 outside the first convex hull 101 (i.e., at more peripheral portions of the transducer arrays) will result in the temperature of the hottest portions of the transducer arrays being monitored. And this can help the system prevent overheating more effectively. Positioning the temperature sensors T1-T4 (e.g., thermistors) outside the first convex hull 101, and, in particular, so that they are not coincident with the metal pads, also enhances the flexibility of the apparatus.

Yet another advantage to this approach relates to the manufacturing process. The thermistors are generally soldered in place and, in the case of polymer coated electrode elements, the soldering typically occurs prior to the polymer coating step. Positioning the thermistors exterior to the polymer layer provides the additional option of positioning and soldering the thermistors after the polymer coating step.

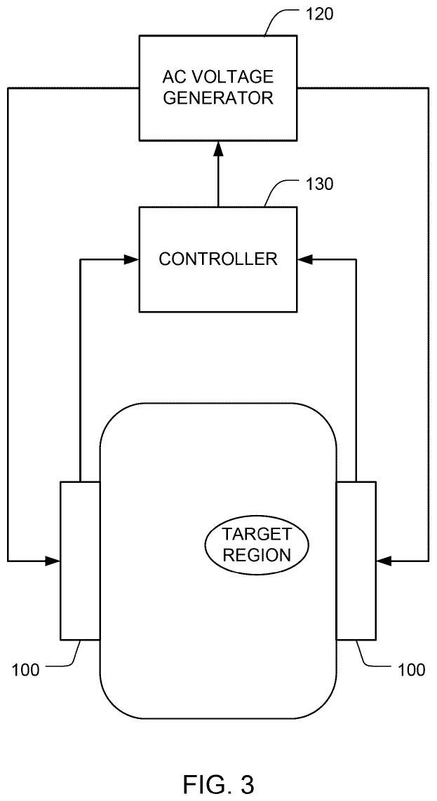

FIG. 3 depicts one example of how to use the apparatus 100 depicted in FIGS. 1-2 to apply alternating electric fields (e.g., TTFields) to a target region in a subject's body. Referring to FIGS. 1-3, a first apparatus 100 is adhered to the subject's skin on one side of the target region, and a second apparatus 100 is adhered to the subject's skin on the opposite side of the target region. To impose alternating electric fields in the target region, the AC voltage generator 120 applies an AC voltage (e.g., between 50 kHz and 5 MHz, or 75-300 kHz) between the metal pads 12 (FIGS. 1-2) of the first apparatus 100 and the metal pads 12 in the second apparatus 100. The AC signal from the AC voltage generator arrives at each of the first and second apparatuses 100 via a set of cables that terminate on the connector 15 of each apparatus 100. And from the connector 15, the signals are routed to the respective metal pads 12 via respective conductive traces (e.g., conductive metal traces, not shown) on the substrate PCB 10.

The controller 130 ascertains the temperature of each of the temperature sensors T1-T4 in each of the first and second apparatuses 100 by inputting respective signals from those temperature sensors. Those signals arrive at the controller 130 via respective conductive traces on the respective substrate PCB 10, respective connectors 15, and respective cables. If the controller 130 determines that all of the temperature sensors are below the threshold temperature, the controller 130 can increase the voltage of the AC signal, which will in turn increase the current that flows through the first and second apparatuses 100, which will in turn increase the intensity of the alternating electric field in the target region. On the other hand, if the controller 130 determines that any of the temperature sensors are at or approaching the threshold temperature, the controller 130 will decrease the voltage of the AC signal, which will decrease the current and eventually decrease the temperature of the hottest region of the apparatus 100.

Notably, the signals that the AC voltage generator 120 sends to the metal pads 12 of the first and second apparatuses 100 have relatively high currents (e.g., on the order of 1 A or even higher). On the other hand, the signals that the controller 130 uses to ascertain the temperature of each of the temperature sensors T1-T4 is orders of magnitude lower (e.g., on the order of 1 mA or even lower). As a result, the substrate PCB 10 can be implemented using relatively thick metal traces (e.g., copper traces) to route the signals from the connector 15 to the metal pads 12, and relatively thin metal traces (e.g., copper traces) to route the signals from the connector 15 to the temperature sensors T1-T4.

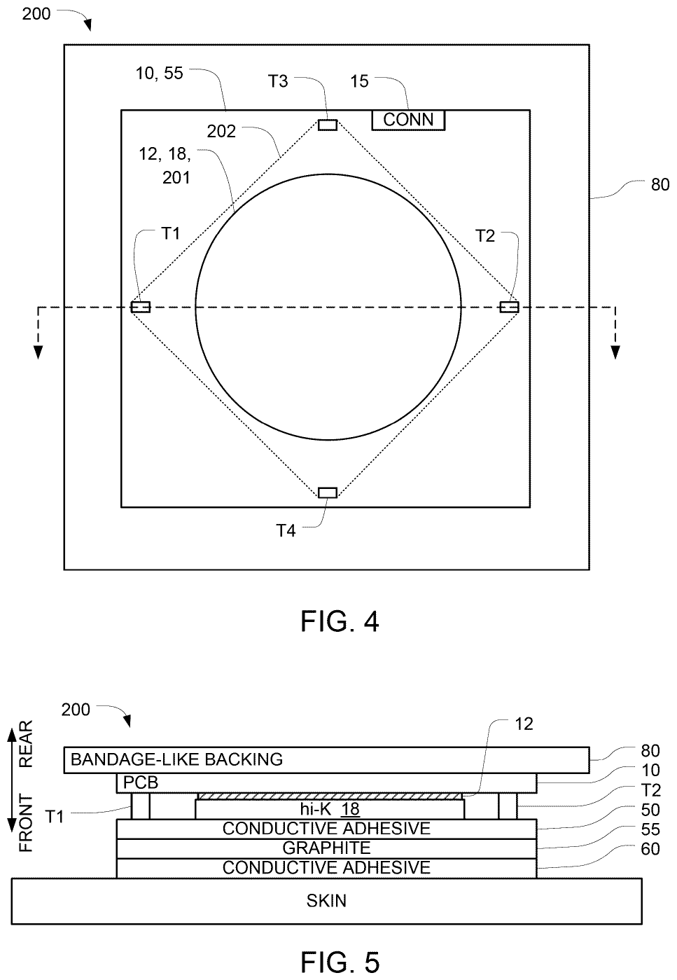

FIG. 4 is a plan view of another apparatus 200 (e.g., a transducer array) for applying alternating electric fields (e.g., TTFields) to a subject's body, and FIG. 5 is a section view of the same apparatus 200. This apparatus is similar to the apparatus 100 described above in connection with FIGS. 1-2 in all respects except that instead of having a plurality of metal pads 12 disposed on the substrate 10, the FIGS. 4-5 embodiment 200 has only a single metal pad 12 disposed on the substrate 10. Notably, when there is only a single metal pad 12 and that pad is convex, the first convex hull 201 (i.e., the convex hull that encloses the single metal pad) will have the same shape as the outline of the single metal pad 12 itself. The second convex hull 202 (i.e., the convex hull that encloses the single metal pad and the plurality of temperature sensors) in the FIGS. 4-5 embodiment has the same shape as the second convex hull 102 in the FIGS. 1-2 embodiment (i.e., the temperature sensors T1-T4 in apparatus 200 are similarly positioned to those in apparatus 100). But note that in other examples the temperature sensors can be positioned at different locations. Other aspects of apparatus 200 (FIGS. 4-5) may be as depicted for the apparatus 100 (FIGS. 1-2) and follow a similar labelling scheme. Note also that not all of the temperature sensors must be positioned outside the first convex hull, as depicted in FIG. 4. To the contrary-some of the temperature sensors can be positioned inside the first convex hull, as long as at least one of the temperature sensors is positioned outside the first convex hull.

The apparatus 200 depicted in FIGS. 4-5 is used to apply alternating electric fields (e.g., TTFields) to a target region in a subject's body in the same way described above in connection with FIG. 3 for the FIGS. 1-2 embodiment.

FIG. 6 is a plan view of another apparatus 300 (e.g., a transducer array) for applying alternating electric fields (e.g., TTFields) to a subject's body, and FIG. 7 is a section view of the same apparatus 300. This apparatus is similar to the apparatus 100 described above in connection with FIGS. 1-2 in all respects except that the shape of the substrate PCB 10 differs dramatically from the shape of the layer of graphite 55 in this FIGS. 6-7 embodiment. More specifically, in this embodiment, the substrate PCB 10 has a main central region that supports the metal pads 12 and is slightly larger than the first convex hull 301 (i.e., the convex hull that encloses all the metal pads 12). And the substrate PCB 10 also has a plurality of stalk-shaped extensions 10x (“protrusions”) that extend from the main central portion. The temperature sensors T1-T4 are mounted on these stalk-shaped extensions 10x (protrusions). The second convex hull 302 (i.e., the convex hull that encloses the metal pads 12 and the temperature sensors T1-T4) in this FIGS. 6-7 embodiment has a shape that is very similar to the second convex hull 102 in the FIGS. 1-2 embodiment. Notably, due to the similar positionings of the metal pads 12 and the temperature sensors T1-T4, this embodiment provides all the advantages of the FIG. 1-2 embodiment described above. And because the PCB substrate 10 is significantly smaller, this embodiment also provides an additional advantage of improved flexibility. Other aspects of apparatus 300 (FIGS. 6-7) may be as depicted for the apparatus 100 (FIGS. 1-2) and follow a similar labelling scheme. Note also that not all of the temperature sensors must be positioned outside the first convex hull, as depicted in FIG. 6. To the contrary-some of the temperature sensors can be positioned inside the first convex hull, as long as at least one of the temperature sensors is positioned outside the first convex hull.

The apparatus 300 depicted in FIGS. 6-7 is used to apply alternating electric fields (e.g., TTFields) to a target region in a subject's body in the same way described above in connection with FIG. 3 for the FIGS. 1-2 embodiment.

FIG. 8 is a plan view of yet another apparatus 400 (e.g., a transducer array) for applying alternating electric fields (e.g., TTFields) to a subject's body, and FIG. 9 is a section view of the same apparatus 400. In the apparatus 400, the substrate of a flexible PCB 10 serves as the substrate. The PCB 10 has at least one metal pad 12 disposed on a front face of the central section of the PCB, and a plurality of conductive traces (not shown) that are also disposed on the PCB. Suitable materials for the at least one metal pad 12 include, e.g., copper and stainless steel. A plurality of temperature sensors (e.g., thermistors) T1-T8 are mounted to the PCB and positioned outside a first convex hull 401 (shown in dotted lines) that encloses the at least one metal pad 12. Each of these temperature sensors T1-T8 is (a) disposed in indirect thermal contact with the at least one metal pad 12 and (b) electrically connected to at least one of the conductive traces of the PCB 10. Note also that not all of the temperature sensors must be positioned outside the first convex hull. To the contrary-some of the temperature sensors can be positioned inside the first convex hull, as long as at least one of the temperature sensors is positioned outside the first convex hull.

Optionally, one or more additional temperature sensors (e.g., thermistors) T9 may be positioned *within* the first convex hull 401. A connector 15 is mounted to the PCB 10, and this connector 15 is used to provide an electrical interface with the thermistors T1-T9 and the at least one metal pad 12. When more than one metal pad 12 is included (as depicted in FIGS. 8-9), all the metal pads 12 can be connected by conductive traces (e.g., metal traces) 13, in which case only a single pin of the connector 15 will be required to apply an AC signal to all of the metal pads 12. In alternative embodiments (not shown), the conductive traces 13 can be omitted, and each metal pad 12 can be electrically connected to its own individual pin on the connector 15.

In the embodiment depicted in FIGS. 8-9, the PCB 10 also includes a layer of dielectric material 18 disposed on and in front of the metal pads 12, and this layer of dielectric material 18 has a dielectric constant of at least 10, and it could be e.g., a polymer film or a layer of a ceramic material. In some preferred embodiments, the layer of dielectric material 18 has a dielectric constant of at least 20 or at least 40. When the layer of dielectric material 18 is present, the indirect thermal contact between the plurality of temperature sensors T1-T8 and the at least one metal pad 12 traverses the layer of dielectric material 18.

A first layer of conductive adhesive 50 is disposed on and in front of the PCB 10. In FIGS. 8-9, the first layer of conductive adhesive 50 is shown disposed on and in front of the PCB 10, and the rear surface of this conductive adhesive 50 adheres to the front surface of the dielectric material 18 and to the front surfaces of the thermistors T1-T8 (and T9, if present) and may also contact some portion(s) of the PCB 10. Note that in some embodiments, the layer of dielectric material 18 is not included, in which case the rear surface of the first layer of conductive adhesive 50 will adhere to the front surface of the metal pads 12 and to the front surfaces of the thermistors T1-T8 (and T9, if present) and may also contact some portion(s) of the PCB 10. Note also that in alternative embodiments, a layer of conductive gel (e.g., hydrogel) can be used in place of the first layer of conductive adhesive 50 depicted in FIG. 9.

A flexible backing 80 (e.g., a bandage-like backing) is positioned behind the PCB 10, and this flexible backing 80 is configured to support the PCB. In the embodiment depicted in FIGS. 8-9, a portion of the flexible backing 80 extends laterally beyond the PCB and the front of this portion may be covered with a biocompatible adhesive that adheres to skin. This portion of the flexible backing 80 helps hold the apparatus 400 against the subject's skin. In other embodiments (not shown), the flexible backing does not extend laterally beyond the substrate PCB 10, and the apparatus 100 may be supported in position on the subject's skin by an adhesive present on the front face of the apparatus (e.g., such as via conductive adhesive 60 or an adhesive coated foam perimeter).

The embodiment depicted in FIGS. 8-9 also includes a layer of anisotropic material (e.g., graphite) 55 that is disposed on and in front of the first layer of conductive adhesive 50, and a second layer of conductive adhesive 60 disposed on and in front of the layer of anisotropic material 55.

The layer of anisotropic material 55 can be a layer of pyrolytic graphite, graphitized polymer film, or graphite foil made from compressed high purity exfoliated mineral graphite. As described above, the layer of anisotropic material 55 has an area that is at least as large as a second convex hull 402 (shown in dotted lines) that encloses the at least one metal pad 12 and the plurality of temperature sensors T1-T8. The layer of anisotropic material 55 facilitates the indirect thermal contact between the plurality of temperature sensors T1-T8 and the at least one metal pad 12. The layer of anisotropic material 55 is preferably both thermally conductive and electrically conductive, in which case it acts to spread both the flow of current and heat in all four directions (i.e., to the right, to the left, into the page, and out of the page in FIG. 9, which corresponds to right, left, up, and down in FIG. 8). In some embodiments, each of the plurality of temperature sensors T1-T8 is positioned near an edge of the layer of anisotropic material. For example, each of the plurality of temperature sensors T1-T8 can be positioned within 2 cm of the edge of the layer of anisotropic material 55, or within 1 cm, or within 5 mm, or within 3 mm of the edge of the layer of anisotropic material 55. (Note that this does not preclude additional temperature sensors e.g., T9 from being positioned at different locations.)

The second layer of conductive adhesive 60 should be biocompatible, and its function is to hold the apparatus 400 against the subject's skin. Note that in alternative embodiments, a layer of conductive gel (e.g., hydrogel) can be used in place of the second layer of conductive adhesive 60 depicted in FIG. 9.

In alternative embodiments of apparatuses described herein (including apparatus 100, 200, 300, 400), the layer of anisotropic material 55 and the second layer of conductive adhesive 60 can be omitted, in which case the first layer of conductive adhesive 50 should be biocompatible so that it can be adhered directly to the subject's skin. Other aspects of apparatus 400 (FIGS. 8-9) may be as depicted for the apparatus 100 or 200 or 300 (FIGS. 1-2, FIGS. 4-5 and FIGS. 6-7, respectively) and follow a similar labelling scheme.

Positioning the temperature sensors T1-T8 outside the first convex hull 401 in the embodiment of FIGS. 8-9 is advantageous for reasons similar to those described above in connection with temperature sensors T1-T4 in the FIG. 1-2 embodiment. And the apparatus 400 depicted in FIGS. 8-9 is used to apply alternating electric fields (e.g., TTFields) to a target region in a subject's body in the same way described above in connection with FIG. 3 for the FIGS. 1-2 embodiment.

In the embodiments described above in connection with FIGS. 1, 2, and 4-9, at least one temperature sensor is positioned outside a convex hull that encloses all the metal pads. But it should be noted that the layouts depicted in those figures are not the only layouts that can be used. To the contrary, there are a wide variety of alternative layouts in which at least one temperature sensor is positioned outside a convex hull that encloses all the metal pads.

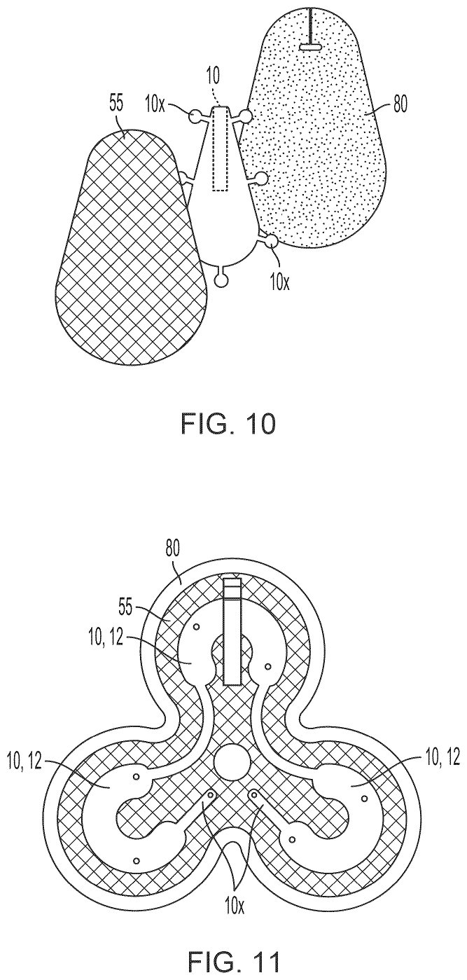

FIG. 10 is an exploded view of one such apparatus. This apparatus is similar to the apparatus 300 described above in connection with FIGS. 6-7 in all respects except that the shapes of the substrate PCB 10, the layer of graphite 55, and the flexible backing 80 are different in this FIG. 10 embodiment. More specifically, in this embodiment, the layer of graphite 55 and the flexible backing 80 are both pear shaped (a stretched asymmetric oval shape). And the PCB 10 has a thin pear shape with a plurality of stalk-shaped extensions (“protrusions”) 10x that extend from a main central portion of the PCB. Temperature sensors (not shown) are mounted on each of these stalk-shaped extensions (protrusions).

FIG. 11 depicts a different apparatus that is constructed in a similar manner to the FIGS. 6-7 embodiment, except for the positioning of the temperature sensors. More specifically, instead of positioning the temperature sensors so that at least one of the temperature sensors is positioned outside a convex hull that encloses all the metal pads 12, the temperature sensors in this FIG. 11 apparatus are positioned at a distal end of at least one of the conductive traces 10x that protrudes from the at least one main portion 10 of the PCB, and the conductive traces to which the temperature sensors are affixed protrude in an inward direction from the at least one main portion of the PCB.

Headings are provided for convenience only and are not to be construed to limit the disclosure in any manner. Embodiments illustrated under any heading or in any portion of the disclosure may be combined with embodiments illustrated under the same or any other heading or other portion of the disclosure. Any combination of the elements described herein and all possible variations thereof is encompassed by the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for applying an electrical signal to a subject's body, the apparatus having a front face facing the subject's body, the apparatus comprising:
a substrate;
at least one metal pad disposed on the substrate;
a plurality of temperature sensors, at least one of which, when viewed from a direction perpendicular to the front face of the apparatus, is disposed on the substrate at a position that is outside a first convex hull that encloses the at least one metal pad; and
a layer of graphite disposed in front of the at least one metal pad and in front of the plurality of temperature sensors, wherein the layer of graphite has an area that is at least as large as a second convex hull that encloses the at least one metal pad and the plurality of temperature sensors, and wherein the layer of graphite is positioned to be in thermal contact with (a) each of the plurality of temperature sensors and (b) the at least one metal pad.

2. The apparatus of claim 1, wherein each of the plurality of temperature sensors is disposed on the substrate at a position that is outside the first convex hull.

3. The apparatus of claim 1, further comprising:
a first layer of conductive adhesive or conductive gel disposed on and behind the layer of graphite; and
a second layer of conductive adhesive or conductive gel disposed on and in front of the layer of graphite.

4. The apparatus of claim 1, further comprising a layer of dielectric material disposed on the at least one metal pad, between the at least one metal pad and the layer of graphite, wherein the thermal contact between the at least one metal pad and the layer of graphite traverses the layer of dielectric material, and wherein the layer of dielectric material has a dielectric constant of at least 10.

5. The apparatus of claim 1, wherein the substrate is a single PCB substrate, wherein the at least one metal pad comprises at least one PCB pad, and wherein the plurality of temperature sensors are mounted to the single PCB substrate.

6. The apparatus of claim 1, wherein the substrate comprises a first PCB having a first PCB substrate and a second PCB having a second PCB substrate, wherein the at least one metal pad comprises at least one PCB pad of the first PCB, and wherein the plurality of temperature sensors are mounted to the second PCB substrate.

7. The apparatus of claim 1, further comprising a flexible backing positioned behind the substrate, wherein the flexible backing is configured to support the substrate.

8. The apparatus of claim 1, wherein the substrate comprises a PCB substrate having one or more central regions, and wherein each of the plurality of temperature sensors is located outside the first convex hull and is disposed on a respective protrusion that extends from the one or more central regions of the PCB substrate.

9. An apparatus for applying an electrical signal to a subject's body, the apparatus having a front face facing the subject's body, the apparatus comprising:
a flexible PCB with at least one metal pad disposed on the PCB and a plurality of conductive traces disposed on the PCB;
a plurality of temperature sensors, at least one of which, when viewed from a direction perpendicular to the front face of the apparatus, is mounted to the PCB and positioned outside a first convex hull that encloses the at least one metal pad, wherein each of the plurality of temperature sensors is (a) disposed in indirect thermal contact with the at least one metal pad and (b) electrically connected to at least one of the conductive traces of the PCB;
a first layer of conductive adhesive or conductive gel disposed on and in front of the PCB; and
a flexible backing positioned behind the PCB, wherein the flexible backing is configured to support the PCB.

10. The apparatus of claim 9, wherein each of the plurality of temperature sensors is mounted to the PCB and positioned outside the first convex hull.

11. The apparatus of claim 9, further comprising:
a layer of anisotropic material disposed on and in front of the first layer of conductive adhesive or conductive gel, wherein the layer of anisotropic material has an area that is at least as large as a second convex hull that encloses the at least one metal pad and the plurality of temperature sensors; and
a second layer of conductive adhesive or conductive gel disposed on and in front of the layer of anisotropic material,
wherein the layer of anisotropic material facilitates the indirect thermal contact between the plurality of temperature sensors and the at least one metal pad.

12. The apparatus of claim 11, wherein the layer of anisotropic material comprises graphite.

13. The apparatus of claim 11, wherein at least one, optionally all, of the plurality of temperature sensors is positioned within 2 cm of an edge of the layer of anisotropic material.

14. The apparatus of claim 11, wherein the flexible PCB has a central region and a plurality of protrusions that extend from the central region, and
wherein the plurality of temperature sensors are mounted on the protrusions.

15. The apparatus of claim 9, wherein the flexible PCB has a central region and a plurality of protrusions that extend from the central region, and
wherein the plurality of temperature sensors are mounted on the protrusions.

16. The apparatus of claim 9, further comprising a layer of dielectric material disposed on the at least one metal pad and in front of the at least one metal pad, wherein the indirect thermal contact between the plurality of temperature sensors and the at least one metal pad traverses the layer of dielectric material, and wherein the layer of dielectric material has a dielectric constant of at least 10.

17. The apparatus of claim 9, wherein the flexible PCB has one or more central regions and one or more protrusions that extend from one or more of the one or more central regions, and
wherein a temperature sensor is mounted on the one or more protrusions.

18. An apparatus for applying an electrical signal to a subject's body, the apparatus having a front face facing the subject's body, the apparatus comprising:
a flexible PCB that includes at least one metal pad positioned on at least one main portion of the PCB, and at least one conductive trace that protrudes from the at least one main portion of the PCB;
a plurality of temperature sensors, at least one of which is (a) disposed in indirect thermal contact with the at least one metal pad, (b) positioned at a distal end of at least one of the conductive traces that protrudes from the at least one main portion of the PCB, and (c) electrically connected to at least one of the conductive traces that protrudes from the at least one main portion of the PCB;
a first layer of conductive adhesive or conductive gel disposed on and in front of the PCB;
a layer of graphite disposed on and in front of the first layer of conductive adhesive or conductive gel, wherein the layer of graphite facilitates the indirect thermal contact between the plurality of temperature sensors and the at least one metal pad;
a second layer of conductive adhesive or conductive gel disposed on and in front of the layer of graphite; and
a flexible backing positioned behind the PCB, wherein the flexible backing is configured to support the PCB.

19. The apparatus of claim 18, wherein at least one of the conductive traces to which a temperature sensor is affixed protrudes in an outward direction from the at least one main portion of the PCB.

20. The apparatus of claim 18, wherein at least one of the conductive traces to which a temperature sensor is affixed protrudes in an inward direction from the at least one main portion of the PCB.
